(19) United States
(12) Patent Application Publication
(10) Pub. No.: US 2025/0285265 A1
(43) Pub. Date: Sep. 11, 2025

(54) SYSTEMS AND METHODS FOR DETECTING STRUCTURES IN 3D IMAGES

(71) Applicant: AFX MEDICAL INC., Montreal (CA)

(72) Inventors: Gabriel CHARTRAND, Montreal (CA); Ramon EMILIANI MEJIA, Montreal (CA); Edward SON, Montreal (CA); Jeremi LAVOIE, Montreal (CA); Simon DUCHARME, Montreal (CA)

(73) Assignee: AFX MEDICAL INC., Montreal, QC (CA)

(21) Appl. No.: 18/860,082

(22) PCT Filed: Apr. 27, 2023
(86) PCT No.: PCT/CA2023/050568

§ 371 Date: Oct. 25, 2024

Related U.S. Application Data
(60) Provisional application No. 63/363,718, filed on Apr. 28, 2022.

(51) Int. Cl. G06T 7/00; G06T 7/136; G06T 15/00; G06V 10/46; G06V 10/82

(52) U.S. Cl. CPC G06T 7/0012; G06T 7/136; G06T 15/00; G06V 10/46; G06V 10/82; G06T 2207/20132; G06T 2207/30016

(57) ABSTRACT

Systems and methods for detecting and contouring structures of interest in a 3D image are provided. In an embodiment, a method includes detecting structures of interest from a multi-channel input comprising at least one 3D image and generating a corresponding segmentation map using a first neural network; extracting a plurality of cropped images from the at least one 3D image, each cropped image corresponding to a subregion of the at least one 3D image containing at least one of the detected structures; and estimating contours of the detected structures in the plurality of cropped images and generating corresponding shape representations of the estimated contours using a second neural network. A corresponding system and non-transitory computer-readable medium are also provided.

### CROSS REFERENCE TO RELATED APPLICATION

The present application claims the benefit of, and priority to, U.S. Provisional Patent Application No. 63/363,718 files Apr. 28, 2022, and entitled “SYSTEMS AND METHODS FOR DETECTING STRUCTURES IN 3D IMAGES”, the entirety of which is incorporated herein by reference.

### TECHNICAL FIELD

The technical field generally relates to 3D imaging, and more specifically to systems and methods for detecting and contouring structures in 3D images to estimate the volume and/or position of the structures.

### BACKGROUND

Brain metastases are increasingly treated using stereotactic radiosurgery. Lesions are precisely targeted by an ionizing radiation beam, destroying target tissue while sparing healthy brain parenchyma to limit cognitive impacts. This treatment requires a planning stage including the identification and delineation of lesions on 3D radiographic images in order for the beam to focus on target areas. Since increasingly complex cases are being treated with this approach, the planning stage imposes a growing burden on radio-oncologists.

There is therefore a need for an automated tool that detect lesions in radiographic images, as well as delineate them and estimate their volume. Such a tool can also be useful for other applications where detecting and contouring small structures of various types in 3D images is required.

Existing image segmentation tools do not give satisfactory results in such tasks for various reasons. The training images usually exhibit a strong imbalance between foreground and background voxels, making cross-entropy loss functions poorly adapted to such a scenario. In some instances, Dice loss functions can be used, but it may introduce other issues. Given the size heterogeneity between structures, smaller structures tend to get ignored in favour of larger ones, reducing the sensitivity to small structures. Moreover, the probability estimates produced by the Dice loss function are poorly calibrated, meaning that the model may be overconfident of its prediction, irrespective of accuracy.

Finally, in existing solutions, the segmentation precision of small structures is limited by the grid resolution of the input image. Structures in the 1 to 5 mm diameter range will be poorly represented by a segmentation mask constrained by a low voxel resolution. This imprecision will tend to propagate when comparing longitudinal scans to assess treatment response.

### SUMMARY

According to an aspect, there is provided a system for detecting and contouring structures of interest in 3D images. The system includes: a computer-implemented neural network module including: a detection module trained to detect the structures of interest from a multi-channel input including at least one 3D image and to generate a corresponding segmentation map of the detected structures; a box sampler configured to extract a plurality of cropped images from the at least one 3D image, each cropped image corresponding to a subregion of the at least one 3D image containing at least one of the detected structures contained in the segmentation map; and a contouring module trained to estimate contours of the detected structures in the plurality of cropped images, and to generate corresponding shape representations.

In an embodiment, the system includes a resampling module configured to resample the 3D image to a configurable resolution.

In an embodiment, the system includes a priors module configured to infer feature descriptors from the at least one 3D image, wherein the multi-channel input further includes the feature descriptors. The feature descriptors can include shape descriptors. Each of the at least one 3D image can be a radiographic image of an organ and the feature descriptors can include anatomical priors.

In an embodiment, each of the at least one 3D image is a radiographic image of a brain and the multi-channel input further includes a brain parenchyma boundary, the system including a brain extraction module configured to infer the brain parenchyma boundary from the at least one 3D image.

In an embodiment, the detection model is configured to output a probability map corresponding to an inferred probability of each voxel of the 3D image being part of one of the structures of interest.

In an embodiment, the detection model is run a configurable number N of times using the same multi-channel input with dropout and data augmentation, outputting N probability maps. The system further includes an aggregation module configured to: compute a mean map and a variance map from the N probability maps; compute a binary map from the mean map; identify components in the binary map, wherein the components define a segmentation and each of the components correspond to one of the detected structures; and aggregate the values of each of the voxels of the variance map corresponding to each of the components of the binary map, wherein each of the aggregated values correspond to a confidence of the corresponding detected structure.

In an embodiment, the detection module and the contouring module each implement a corresponding convolutional neural network.

In an embodiment, the system further includes an interactive validation module, the interactive validation module being configured to: for each of the at least one detected structures, display the detected structure, allow for the approval or rejection of the detected structure, and allow for the modification of the contour corresponding to the detected structure; and allow for the request of an additional structure, including the contouring module estimating an additional contour corresponding to the additional structure to generate a corresponding shape representation.

In an embodiment, the system further includes a reporting module configured to produce a report of the detected structures.

In an embodiment, the system further includes a picture archiving and communication system (PACS); an import module configured to acquire the at least one 3D image from a plurality of DICOM files on the PACS; and an export module configured to export each of the implicit shape representation to a plurality of DICOM-RT files on the PACS.

In an embodiment, the system further includes a volume estimation module configured to estimate a volume for each of structures of interest from the corresponding structure contour definition.

In an embodiment, the structures of interest correspond to brain metastases.

According to another aspect, there is provided a computer-implemented method for training a neural network to detect and contour structures of interest in 3D images, the method including: training a detection model to detect the structures of interest from a multi-channel input including at least one 3D image and to generate corresponding segmentation map of the detected structures; and training a contouring model to estimate contours of structures to generate corresponding shape representations.

In an embodiment, the method further includes receiving a training dataset including the at least one 3D images and a corresponding plurality of sets of ground truth contour definitions, each of the contour definitions corresponding to one of the structures of interest.

In an embodiment, each of the at least one 3D image is a radiographic image of a brain and the multi-channel input further includes a brain parenchyma boundary, the method further including inferring a brain parenchyma boundary from each of the plurality of 3D images.

In an embodiment, the method further includes rasterizing the plurality of sets of ground truth contour definitions.

In an embodiment, the method further includes resampling the plurality of 3D images and the plurality of sets of ground truth contour definitions.

In an embodiment, the method further includes inferring feature descriptors from each of the plurality of 3D images, wherein the multi-channel input further includes the feature descriptors.

In an embodiment, the method further includes sampling the plurality of 3D images and the plurality of sets of ground truth contour definitions.

In an embodiment, the sampling includes: selecting a plurality of voxels from each of the plurality of 3D images; and for each of the plurality of voxels, extracting a corresponding patch centred around the voxel.

In an embodiment, the selecting the plurality of voxels includes, for each of the structures of interest, selecting one voxel within the structure of interest.

In an embodiment, the training the detection model includes using a modified Dice loss function.

In an embodiment, the modified Dice loss function is a structure-wise Dice loss function and includes a focal-like weight.

In an embodiment, the training the detection model includes using dropout.

In an embodiment, the training the detection model includes using a first modified stochastic gradient descent algorithm.

In an embodiment, the method further includes, for each one of the structures of interest from the plurality of 3D images and the corresponding plurality of sets of contour definitions, a cropped bounding box centred around the structure of interest.

In an embodiment, the training the contouring model includes using a first channel corresponding to the cropped bounding box.

In an embodiment, the training the contouring model includes using a second channel corresponding to a ground truth segmentation.

In an embodiment, the second channel is randomly dropped.

In an embodiment, each of the structure contour definitions inferred by the contouring models and each of the ground truth contour definitions correspond to a levelset.

In an embodiment, each of the levelsets correspond to a distance map.

In an embodiment, the training the contouring model includes using a modified L₂ loss.

In an embodiment, the modified L₂ loss is clipped with respect to a threshold.

In an embodiment, the training the detection model includes using a second modified stochastic gradient descent algorithm.

In an embodiment, the structures of interest correspond to brain metastases.

According to an aspect, a method for detecting and contouring structures of interest in a 3D image in provided. The method includes: detecting structures of interest from a multi-channel input comprising at least one 3D image and generating a corresponding segmentation map of the detected structures using a first neural network; extracting a plurality of cropped images from the at least one 3D image, each cropped image corresponding to a subregion of the at least one 3D image containing at least one of the detected structures contained in the segmentation map; and estimating contours of the detected structures in the plurality of cropped images and generating corresponding shape representations of the estimated contours using a second neural network.

According to an aspect, a computing system for detecting and contouring structures of interest in a 3D image is provided. The computing system includes one or more processors and memory, the memory having instructions stored thereon which, when executed by the one or more processors, cause the computing system to: detect structures of interest from a multi-channel input comprising at least one 3D image and generate a corresponding segmentation map of the detected structures using a first neural network; extract a plurality of cropped images from the at least one 3D image, each cropped image corresponding to a subregion of the at least one 3D image containing at least one of the detected structures contained in the segmentation map; and estimate contours of the detected structures in the plurality of cropped images and generate corresponding shape representations of the estimated contours using a second neural network.

According to an aspect, a non-transitory computer-readable medium is provided. The computer-readable medium has instructions stored thereon which, when executed by one or more processors of a computing system, cause the computing system to: detect structures of interest from a multi-channel input comprising at least one 3D image and generate a corresponding segmentation map of the detected structures using a first neural network; extract a plurality of cropped images from the at least one 3D image, each cropped image corresponding to a subregion of the at least one 3D image containing at least one of the detected structures contained in the segmentation map; and estimate contours of the detected structures in the plurality of cropped images and generate corresponding shape representations of the estimated contours using a second neural network.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment.

FIG. 1 is a schematic of a system for detecting and contouring structures of interest in 3D images, according to an embodiment.

FIG. 2 is a schematic of a medical imaging and treatment planning system comprising a system for detecting and contouring structures of interest in 3D images.

FIG. 3 is a schematic of a preprocessing system for use with a system for detecting and contouring structures of interest in 3D images.

FIG. 4 is a schematic of a system for detecting and contouring structures of interest in 3D images, according to an embodiment that uses augmentation and dropout.

FIGS. 5A and 5B are an example of intermediary outputs of the embodiment shown in FIG. 4.

FIG. 6 is a schematic of a system for detecting and contouring structures of interest in 3D images, according to an embodiment including an interactive validation module.

FIGS. 7A and 7B are a schematic of a method for training neural networks used in a system for detecting and contouring structures of interest in 3D images, according to an embodiment.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practised without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

As will be appreciated, the present disclosure has broad applications in fields working with 3D images where there is an interest in detecting and contouring structures within those 3D images in order to precisely estimate the volume and/or position of the structures. The present disclosure provides examples and embodiments where brain metastases are detected and contoured in images captured by magnetic resonance imaging (MRI) machines to plan a stereotactic radiosurgery, where lesions are precisely targeted by an ionizing radiation beam, destroying target tissue while sparing healthy brain parenchyma to limit cognitive impacts, or to follow up on a treatment. It is appreciated, however, that this if for exemplary purposes only and that the present disclosure can apply to detecting and/or contouring different structures within MRI or other types of 3D images.

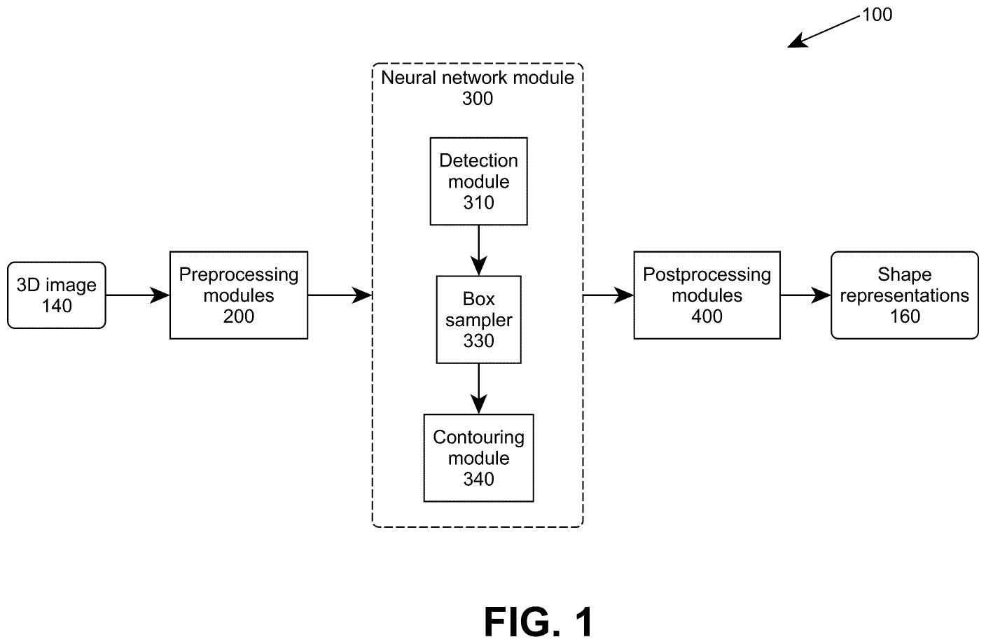

With reference to FIG. 1, an exemplary system 100 to detect and contour structures of interest in 3D images is shown. Broadly described, the system 100 comprises a neural network module 300 configured to receive a tensor representing at least a 3D image 140 as an input and to produce therefrom shape representations 160 comprising the contour of structures of interest detected by the neural network module 300 in the 3D image 140. It will be appreciated that the described system can be used to carry out a corresponding method for detecting and contouring structures of interest in a 3D image.

In some embodiments, various preprocessing modules 200 can be configured to prepare the 3D image 140 before it is provided as input to the neural network module 300 and/or to infer parameters from that can be provided as additional input to the neural network module 300. In some embodiments, the output of the neural network module 300 can be an implicit representation of the shape of the detected structures, such as for instance a distance map. In these and other embodiments, various postprocessing modules 400 can operate on the output in order to modify the output, for instance changing it to an explicit representation such as an isocontour, to infer additional parameters from the output, and/or to implement manual validation steps and/or reporting steps.

The neural network module 300 comprises one or more neural networks trained on corresponding tensors comprising 3D images with a corresponding ground truth indicating the contours of structures of interest, such that the network module 300 is trained to detect and contour the structures. In some embodiments, additional submodules can be provided in the neural network module 300, for instance to facilitate using the output of one neural network as the input of another neural network. It is understood that the neural networks and additional submodules can be implemented using computer hardware elements, computer software elements or a combination thereof. Accordingly, the neural networks and additional submodules described herein can be referred to as being computer-implemented. Various computationally intensive tasks of the neural network can be carried out on one or more processors (central processing units and/or graphical processing units) of one or more programmable computers. For example, and without limitation, the programmable computer may be a programmable logic unit, a mainframe computer, server, personal computer, cloud-based program or system, laptop, personal data assistant, cellular telephone, smartphone, wearable device, tablet device, virtual reality device, smart display devices such as a smart TV, set-top box, video game console, or portable video game device, among others.

In the exemplary detection and contouring system 100, the neural network module 300 comprises an architecture that includes at least two separate modules, namely a detection module 310 and a contouring module 340, and an additional submodule, namely a box sampler 330, that converts the output of the detection module 310 into a form suitable to be used as input by the contouring module 340. Each of the detection module 310 and the contouring module 340 can implement neural respective networks that are based on a convolution neural network (CNN). As can be appreciated, CNNs are suitable for analyzing imagery given that they are space or shift invariant, although it is appreciated that other types of neural networks are also possible.

The detection module 310 can be configured to take as input a multi-channel input including a tensor comprising at least a 3D image 140 and produce as output a probability map where the value assigned to each voxel represents the probability that this voxel belongs to a structure of interest. The probability map along with a configurable threshold corresponds to a segmentation map of the detected structures. In some embodiments, the detection module 310 can implement a sliding window algorithm, wherein the 3D image 140 is sampled into patches in a grid-like fashion, such that each patch represents a region of the 3D image 310 that can at least partially overlap with regions represented in other patches, for instance by a factor of 50%. Each patch can therefore be fed as input to a first neural network implemented by the detection module 310, generating a plurality of probability maps that can then be aggregated into a single probability map. This approach can allow for the detection module 310 to process incrementally larger images using constant memory.

In the illustrated embodiment, the probability map is provided as input to box sampler 330. Voxels of the 3D image 310 whose corresponding entry in the probability map is above a configurable threshold define a segmentation of the detected structures of interest. For each detected structure of interest, the box sampler can be configured to extract a bounding box, or “bbox”, from the 3D image which contains the detected structure. For example, the bounding box can be centred and cropped around each region of the 3D image corresponding to a detected structure, and adjusted to a zoom level such that the bounding box has a specific size, for instance 128³ voxels. In some embodiments, the detected structure can be surrounded by a margin within the bounding box, for instance of 20 voxels.

Each bounding box is provided as input to the contouring module 340, which produces an output representative of the shape of the structure contained within the bounding box, for example using a second neural network implemented via contouring module 340. In the present embodiment, the output comprises an implicit shape representation, although it is appreciated that other shape representations are possible. The implicit shape representation can for instance be as a levelset image, or an isocontour map, where each voxel is assigned a value representing the signed distance from the boundary of the detected structure. The boundary can be implicitly represented by the zero-crossing location within the isocontour map.

As can be appreciated, the 3D image 140 can correspond to a 3D image acquired via an image acquisition device. As an example, the image can be a radiographic image, such as a magnetic resonance imaging (MRI) brain scan, although it is appreciated that other 3D images are possible such as those acquired via electronic microscopy (EM), industrial computed tomography (CT), or other techniques for acquiring 3D images of biological or non-biological specimens. The 3D image can be received in various different formats, such as the Digital Imaging and Communications in Medicine (DICOM) format in the case of medical images, such as 3D radiographic images. In some embodiments, the 3D image can be received as a plurality of 2D slices, and the 3D image can be reconstructed therefrom.

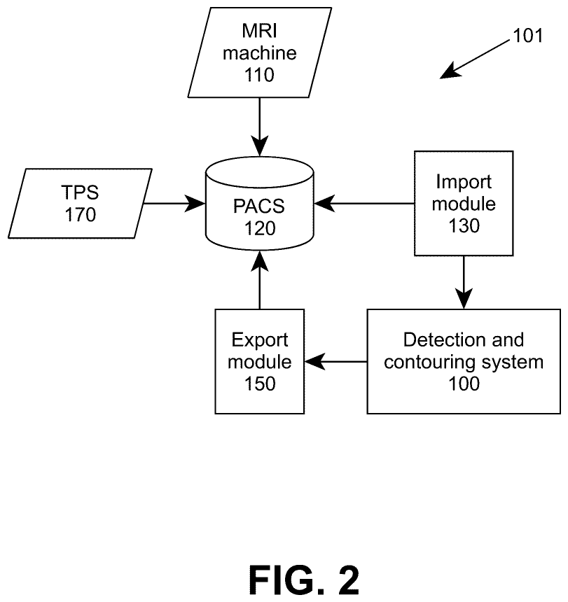

With reference to FIG. 2, an embodiment of a medical imaging and treatment system 101 comprising the detection and contouring system 100 is shown. In the illustrated embodiment, the 3D image 140 can be acquired from an MRI machine 110 and stored as DICOM files in a picture archiving and communication system (PACS) 120. The PACS 120 can for instance be implemented in a hospital and comprise imaging modalities, such as MRI machines and CT machines, archiving devices configured to store radiographic images acquired by the modalities in addition to reports and patient information. The PACS 120 can further include workstations running applications configured to provide medical personnel access to the stored images and information, and be supported by a network infrastructure, including for instance a local area network, a wide area network such as the Internet and/or a virtual local area network securely deployed for instance over the Internet.

The illustrated system 101 can be configured to detect small lesions in radiographic images, for instance brain metastases in T1-weighted brain MRI images with contrast agent, which can be used to assist in the planning of stereotactic radiosurgery (SRS) on a treatment planning system 170 (TPS). The MRI images archived in the PACS 120 as DICOM files representing a plurality of 2D slices can be acquired by an import module 130. Either the import module 130 or a distinct converter module can be configured to reconstruct a 3D image therefrom for consumption by the detection and contouring system 100. The shape representations generated by the detection and contouring system 100 can then be converted by an export module, for instance to DICOM-RT files for archiving in the PACS 120. Once archived in the PACS 120, the DICOM-RT files can be accessed by a TPS 170 in order to plan radiosurgery.

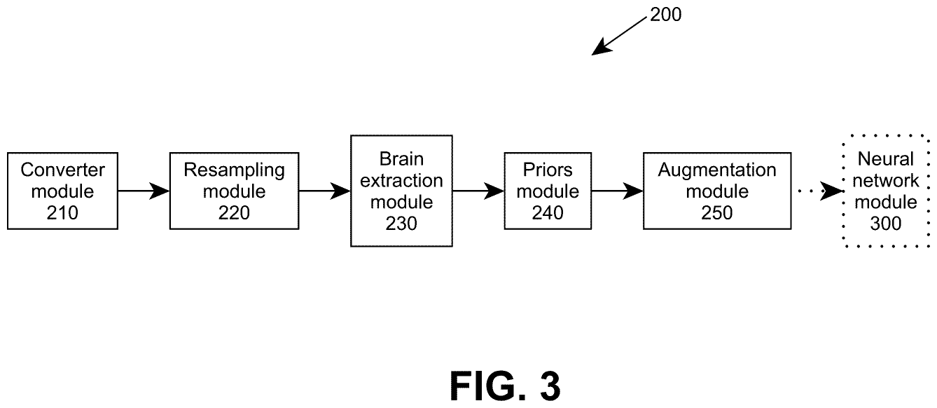

With reference to FIG. 3, and as explained above, various preprocessing modules 200 can operate imported images in order to modify and prepare 3D images before being provided as input to the neural network module 300 and/or to infer parameters that can be provided as additional input to the neural network module 300. It should be appreciated that the illustrated sequence of modules 200 is provided for exemplary purposes only, and that in other embodiments the modules 200 can be arranged differently to apply preprocessing steps in a different order.

In some embodiments, a converter module 210 can be provided to convert imported images into a volumetric representation. For example, in embodiments where the imported images correspond to 2D slices, the converter module 210 can be configured to convert the slices to a 3D image useable by the neural network module 300.

In some embodiments, a 3D image may be acquired as a plurality of 2D slices, requiring a converter module 210 configured to convert the slices to a volumetric representation useable by the neural network module 300.

A resampling module 220 can thereafter be used to resample the image to a configurable resolution, for instance an isotropic resolution where each voxel side corresponds to a width of 1 mm.

In medical applications, such as in the system 101 of FIG. 2, the 3D image can correspond to an MRI brain scan. In such an embodiment, the performance of the neural network module 300 can be improved by incorporating into the input tensor a map that represents an anatomical segmentation of the brain, for instance a map where each coordinate corresponding to a voxel outside the brain parenchyma is assigned a value of 0, computed by a brain extraction module 230. Different approaches and tools for locating the brain parenchyma boundary in MRI brain scans can be used, such as brain extraction tools or skull strippers. For instance the approaches that can be used can include those described in Smith, S. M. (2002); Fast robust automated brain extraction; Human brain mapping , 17(3), 143-155, and in Iglesias, J. E., Liu, C. Y., Thompson, P. M., & Tu, Z. (2011); Robust brain extraction across datasets and comparison with publicly available methods; IEEE transactions on medical imaging, 30(9), 1617-1634, the disclosures of which are incorporated herein by reference in their entireties.

The performance of the neural network module 300 can be further improved by incorporating into the input tensor one or more feature maps corresponding for instance to feature descriptors of elements of the 3D image, comprising for instance shape descriptors and anatomical priors, computed by a priors module 240. For instance, shape descriptors can include tubularity and sphericity descriptors. In medical applications, these can be supplemented by one or more feature maps that represent anatomical priors, for instance an anatomical segmentation of the imaged organ, including for instance blood vessels, which may otherwise appear as lesions in detection models working with contrasted radiographic images. While an additional neural network, for instance a CNN, can be trained to infer shape descriptors and anatomical segmentations, it is possible to use other approaches, such as those described for instance in Frangi, A. F., Niessen, W. J., Vincken, K. L., & Viergever, M. A. (1998); Multiscale vessel enhancement filtering; International conference on medical image computing and computer-assisted intervention, 130-137 and Krissian, K., Malandain, G., Ayache, N., Vaillant, R., & Trousset, Y. (2000); Model-based detection of tubular structures in 3D images; Computer vision and image understanding, 80(2), 130-171, the disclosures of which are incorporated herein by reference in their entireties.

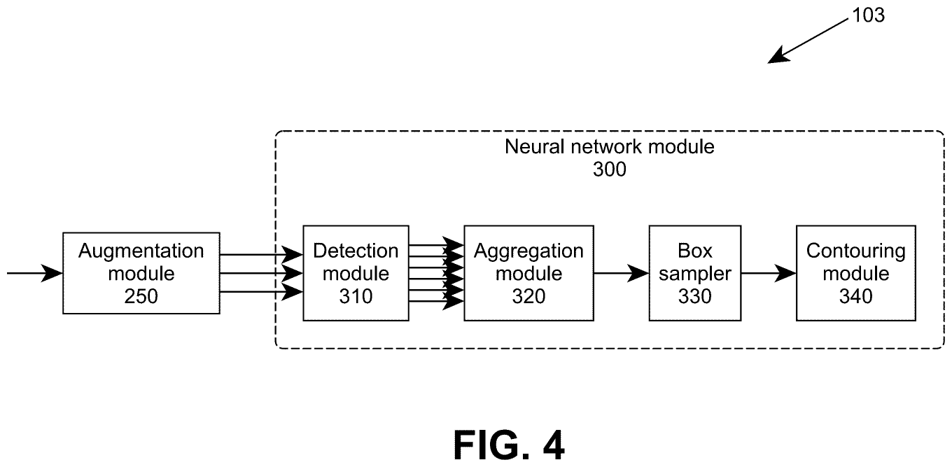

In some embodiments, the accuracy of probability map generated by the detection module 310 can be improved by introducing perturbations in the 3D image provided as input to the neural network 300. In the present embodiment, such perturbations are applied via an augmentation module 250. The augmentation module 250 can be configured to generate a plurality of 3D images from an original 3D image by applying one or more perturbations to the original 3D image. The perturbation can, for example, be applied using processes similar to those used for data augmentation at training-time. For instance, some generated 3D images can be flipped with respect to the original and/or have random gaussian noise added or gamma contrast correction applied, among other perturbations. As shown in the exemplary system 103 of FIG. 4, each of the plurality of 3D image generated by the augmentation module 250 can be fed separately to the detection module 310 as part of its input. In some embodiments, the detection module 310 can implement random dropout in its neural network model at testing time, effectively simulating using a plurality of neural network models having the same architecture but different parameters.

As can be appreciated, using the augmentation module 250 and/or dropout in detection module 310 results in the detection module 310 outputting a plurality of slightly different probability maps. The probability maps can be aggregated by an aggregation module 320 such that a single probability map is provided as an input to the box sampler 330. The consensus between the plurality of prediction maps can be used as a proxy for the model confidence.

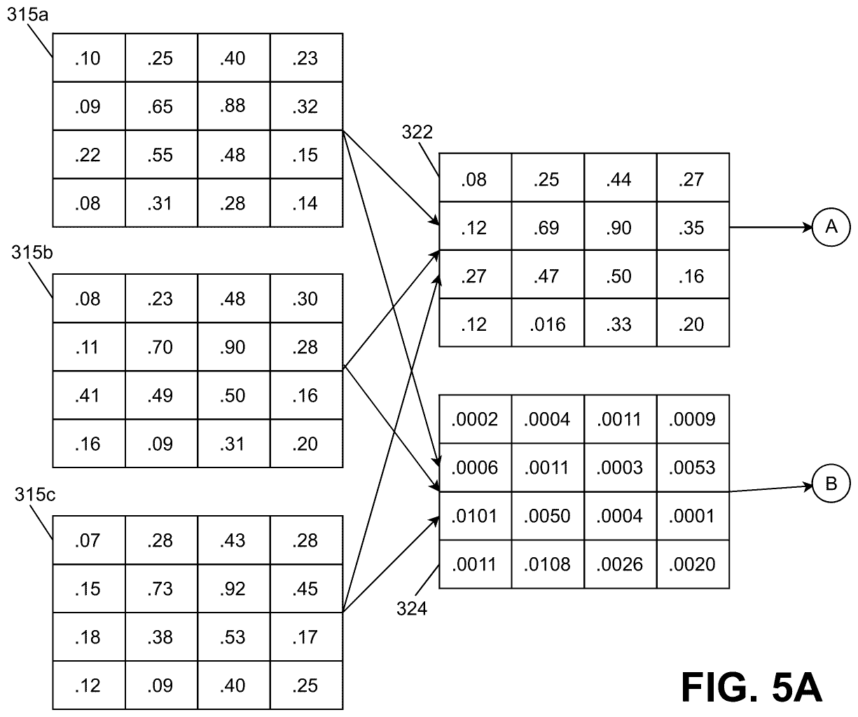

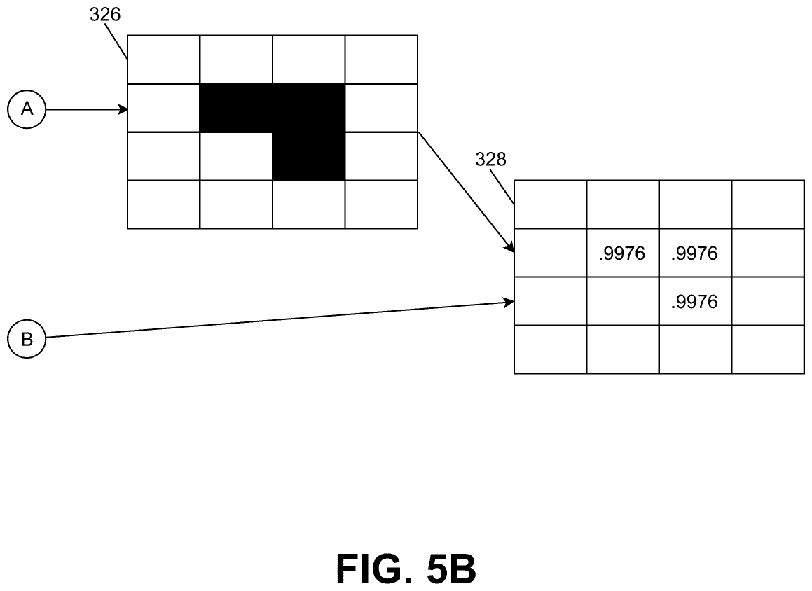

As an example, referring to FIGS. 5A and 5B, the aggregation module 320 can be configured to compute a mean map 322 from the plurality of probability maps 315a, 315b and 315c received from detection module 310, wherein each coordinate of the mean map 322 has a value equal to the mean of the corresponding values of the probability maps 315a, 315b and 315c. The aggregation module can be further configured to compute a variance map 324, wherein each coordinate of the variance map 324 has a value equal to the variance of the corresponding values of the probability maps 315a, 315b and 315c.

The mean map 322 can be used to generate a binary map 326 by applying a threshold, for instance 0.5, such that each coordinate in the mean map 322 with a probability mean equal to or greater than the threshold has a corresponding value in the binary map 326 indicating the that the detection module 310 has identified a structure of interest in the corresponding voxel.

A connected component transform can be applied to the binary map 326 to identify graph-theoretical components among the voxels, with each component corresponding to a detected structure.

The variance map 324 can be used to compute, for all the voxels that are part of each detected structure according to the binary map 326, an aggregation of the variances of the structures 328, which can be provided as an input to the box sampler 330. This aggregated variance can be used to compute a confidence score for the detected structure. The aggregation function of the variance can be chosen such that detected structure exhibits a calibrated score, meaning that for example, a confidence of 30% will lead to correct detection 3 out 10 times. As an example, the aggregation can be the mean, for all voxels that are part of a detected structure, of the result of the calculation

$$1 - \frac{{σ}^{2}}{0.25} .$$

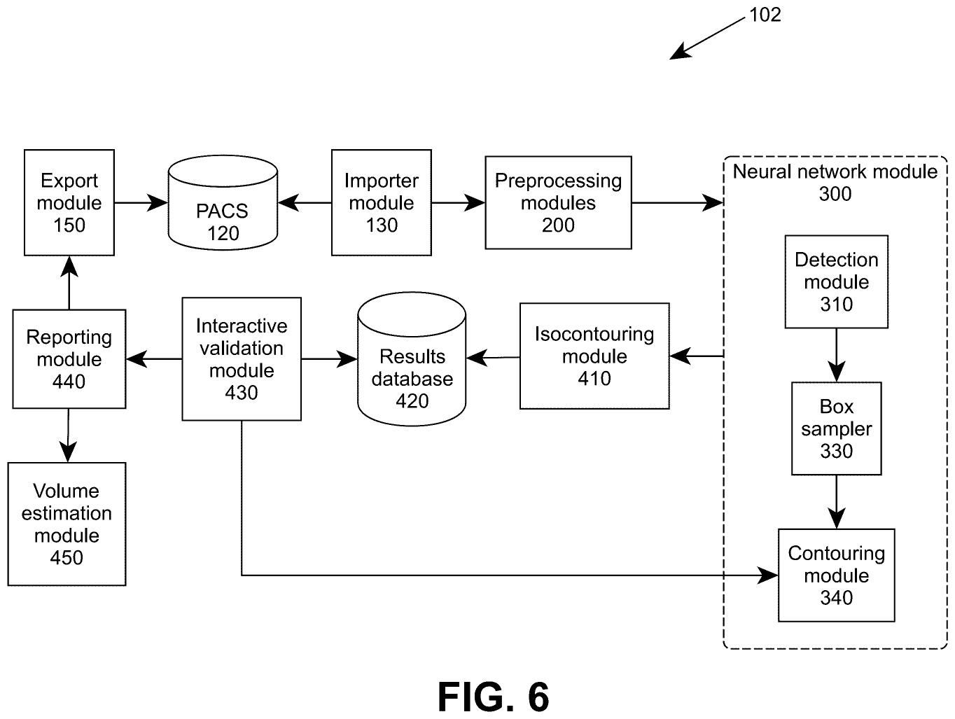

As explained above, various postprocessing modules can operate on the output. In some embodiments, the output of the contouring module 340 is an implicit shape representation such as an isocontour map. With reference to FIG. 6, an explicit contour representation, for instance a polyline or a polygonal surface, can be recovered from an implicit representation by an isocontouring module 410 using an iso-surface algorithm such as marching cubes or marching squares. In some embodiments, the shape can be adjusted by changing the threshold applied to extract the surface, allowing the extraction of a larger version of a detected structure while preserving its overall shape, for instance if the boundary is imprecise and/or fuzzy.

In the embodiment of a system 102 for detecting and contouring structures of interest in 3D images shown in FIG. 6, an interactive validation module 430 is provided to allow users to make modifications to the detected and contoured structures before they are committed, for instance by the export module 150 to the PACS 120. Because DICOM files are meant to be immutable, the output of the neural network module 300 can be stored temporarily in an alterable format outside the PACS, for instance in a results database 420. The list of detected structures can be presented to users, for example via a graphical user interface (GUI) of a user device. The structures can, for instance, be sorted by confidence score. Controls can be provided via the GUI to allow the user to provide input via the user device to approve or reject each structure. In some embodiments, controls can be provided via the GUI to allow the user to interactively modify the contour boundary location by changing the isocontour value of the implicit shape contour representation, or altering the implicit shape representation directly by manually placing boundary constraints. In some embodiments, controls can be provided via the GUI to allow the user to furthermore request that a new structure be added by manually defining a bounding box, which is then fed to the contouring module 340 and added to the results. Finally, the user can approve the list of structures and their contours, which can then as an example be converted to a polyline representation and saved in the PACS 120 as DICOM-RT files by the export module 150.

As can be appreciated, the interactive validation module 430 can be configured to generate the GUI in the form of a web page consisting of code in one or more computer languages, such as HTML, XML, CSS, JavaScript and ECMAScript. In some embodiments, the GUI can be generated programmatically, for instance on a server hosting the results database 420, and rendered by an application such as a web browser on a user device, such as a workstation. In other embodiments, the interactive validation module 430 can be configured to generate the GUI via a native application running on the user device, for example comprising graphical widgets configured to render information received from the results database 420.

In some embodiments, a reporting module 440 can be provided to generate a report that indicates various parameters relating to detected structures, such as their location, volume, diameter and identifier, among others. A volume estimation module 450 can use the explicit shape representation generated by the isocontouring module 410 to precisely compute the diameter and the volume of the structure, which can be included in the report. In some embodiments, the report can be stored along with corresponding DICOM-RT files in the PACS 120 via the export module 150.

As will be described in more detail hereinafter, the detection module 310 and the contouring module 340 can be trained independently from one another to produce segmentation maps and shape representations. The neural network module 300 can be trained (i.e. fitted, regressed) in two stages. The detection module 310 can be trained to produce probability maps that can be translated into low-resolution segmentation maps from 3D images and, optionally, anatomical segmentations of the brain and/or feature descriptors. The contouring module 340 can be trained to produce precise, high resolution shape representations from cropped 3D images corresponding to segmentation maps obtained from the detection module 340. It can be appreciated that training the detection module 310 and contouring module 340 can be carried out in any order, or in parallel. At inference time, both modules 310 and 340 can be connected together to produce a complete segmentation of structures of interest in a 3D image in a single pass.

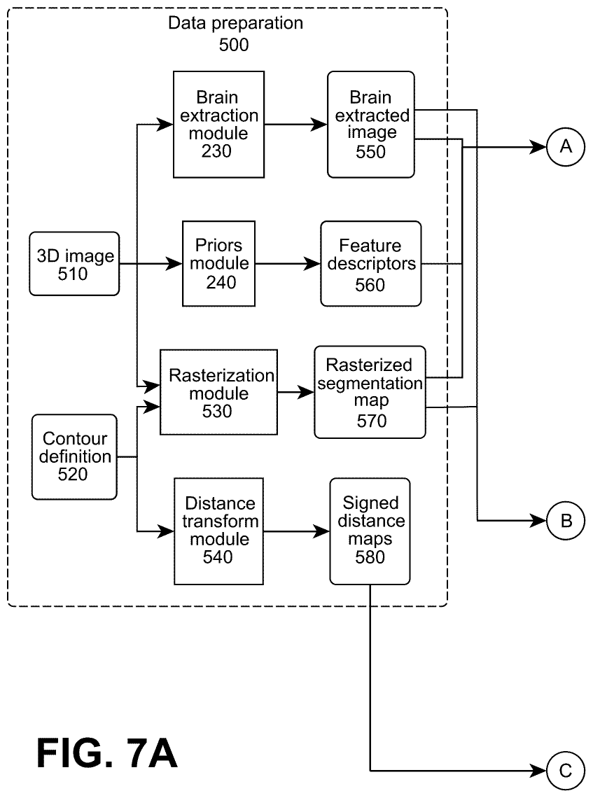

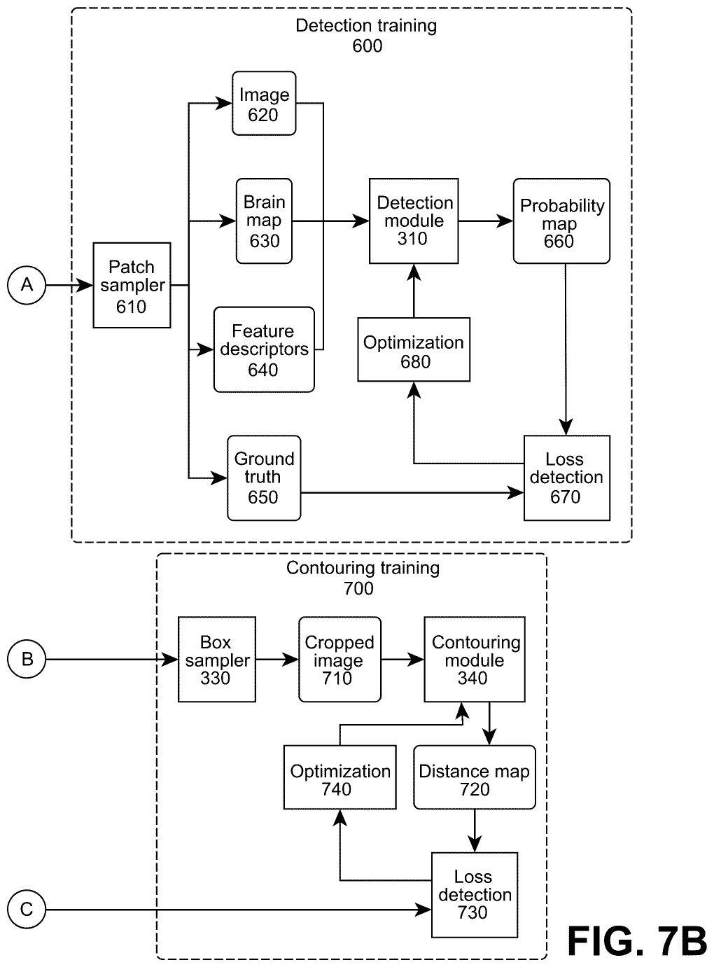

With reference to FIGS. 7A and 7B, an exemplary method of training neural networks used in a system for detecting and contouring structures of interest in 3D images is shown according to an embodiment. The method includes a data preparation phase 500 to prepare training data, a detection training phase 600 to train the detection module, and a contouring training phase 700 to train the contouring module. As will be appreciated, the neural network module 300 can be trained via supervised learning techniques. Accordingly, to train the neural network module 300, a training dataset comprising a plurality of 3D image with corresponding ground truth contour definitions (i.e. structure segmentations) can be provided. The plurality of images can have the same photometric profiles (e.g. all T1-weighted images), have known spacing in each dimension, account for variations encountered in real life, be collected from a plurality of different centres or studies, and be significant in number (e.g., in the hundreds). Moreover, the plurality of images can all correspond to images of the same object and/or anatomical structure to which the neural network is to be trained/fitted.

In some embodiments, the images and segmentations can be received from one or more external data sources, such as from one or more open health databases. As one skilled in the art would appreciate, open health databases can include a plurality of 3D medical images acquired via one or more medical imaging devices, at one or more different locations, by one or more different parties, and/or in one or more different contexts (for example as part of one or more different studies). The received training dataset can include contours of structures of interest in each 3D image. The contours can correspond to contours drawn manually, inferred via one or more existing algorithms, and/or manually validated. Each contour can thus be taken as an accurate representation of the structures in the 3D image to which it corresponds and can therefore be used as a ground truth to train the neural network module 300.

As an example, a training dataset of T1-weighted brain MRI scans with contrast agent such as gadolinium can be assembled. Each scan can show a plurality of lesions, for instance metastases, and have associated DICOM-RT files containing coordinates for series of polylines, corresponding to ground truth contour definitions delineating target structures and/or lesions, on each slice of the scan. These types of images and contours can be acquired and produced in the course of radiotherapy treatment planning and follow-up, for example.

In some embodiments, each 3D image 510 and corresponding contour definition 520 can be prepared prior to their use for training, for instance as part of the illustrated data preparation phase 500. In the illustrated embodiment, the brain extraction and priors modules 230 and 240 described above can be respectively applied to the 3D image 510 to obtain a corresponding brain extracted image 550 and corresponding feature descriptors 560. A rasterization module 530 can be applied to the contour definition 520 in accordance with the parameters of the 3D image 510 to obtain a rasterized segmentation map, representing the contours. The coordinates corresponding to a voxel within the contour of a structure of interest, which can be referred to as foreground voxels, can be assigned a positive value, while coordinates corresponding to a voxel not in a structure of interest, which can be referred to as background voxels, can be assigned a zero value. Furthermore, in embodiments where the ground truth contour definitions 520 are not represented by signed distance maps 580, they can be converted to one by a distance transform module 540 implementing for instance an interpolation algorithm, such as a radial basis function interpolation algorithm, or a signed distance transform. A resampling module 220 can thereafter be used to resample the generated maps 550, 560, 570 and 580 to the same configurable resolution used for instance in the exemplary detection and contouring system 100, for instance an isotropic resolution where each voxel side corresponds to a width of 1 mm.

As part of the detection training phase 600, the brain extracted image 550, the feature descriptors 560 and the rasterized segmentation map 570 can be provided as an input to a patch sampler 610. The patch sampler 610 can be configured to sample or extract a plurality of cropped samples or patches of suitable size from the 3D image 510, for instance of size 128³, and to recreate an image 620, a brain map 630, feature descriptors 640 and a ground truth 650 corresponding to each cropped sample.

In some embodiments, the patch sampler 610 can be configured such that the centre location of the patch is randomly sampled from foreground and background voxels, such that at least 50% of the generated patches contain at least one structure of interest. However, it can be appreciated that when the structure of interest distribution in the training dataset exhibits size heterogeneity, larger structures can be exponentially more susceptible to being sampled. Accordingly, in other embodiments, the patch sampler 610 can first sample an individual structure, then sample a voxel within that structure, ensuring that each structure has an equal chance of being sampled, irrespective of its size.

As an example, a volume-aware sampling strategy can be applied where a sampling map M can be defined such that each foreground voxel has a probability of being sampled inversely proportional to the volume of the structure it belongs to. This map can be constructed by first assigning a probability p_{bg} of sampling a background voxel. The remaining probability mass can be split uniformly among n individual structures produced by a connected-component transform, such that Pᵢ=(1−P_{bg})/n is the probability of sampling any structure i. Finally, the probability assigned to individual voxels q of a structure i can be the structure's probability over the structure's volume, such that Mᵢ(q)=pᵢ/vol (i), where vol(i) is the volume of structure i. The resulting sampling map can be expressed as

$$M ( q ) = \{ \begin{matrix}\frac{( 1 - {p}_{bg} ) / n , if q ∈ {g}_{CC}^{i}}{vol ( i )} \\ \frac{{p}_{bg}}{vol ( bg )} , otherwise\end{matrix}$$

where g_{CC}ⁱ is the voxel set belonging to structure i, n is the number of distinct connected components, and vol(i) and vol(bg) are respectively the volumes of structure i and background voxels. For both sampling strategies, p_{bg} can be set to 0.5, such that at least half of the patches contain a structure. Patches can then constructed by sampling a centre voxel from the sampling map M and cropping a bounding box around that centre voxel. It can be appreciated that different sampling strategies can be implemented for datasets exhibiting different characteristics.

In some embodiments, in particular when training data is limited, random image augmentations can be applied on each full-sized volume before patches are sampled, in order to create additional training images and increase the robustness of the trained detection module 310. Augmentations can comprise for instance randomly flipping images, adding random Gaussian noise, applying gamma contrast correction and/or introducing motion artifacts and/or bias field distortions. As an example, random Gaussian noise with a mean of zero and a standard deviation between 0 and 0.25 along with a gamma contrast correction of (−0.3, 0.3) can be applied to 50% of the images, motion artifacts can be introduced in 10% of the images and bias field distortions can be introduced in 25% of the images, using tools such as TorchIO, as described for instance in Perez-García, F., Sparks, R., & Ourselin, S. (2021); TorchIO: a Python library for efficient loading, preprocessing, augmentation and patch-based sampling of medical images in deep learning; Computer Methods and Programs in Biomedicine, 208, 106236, the disclosure of which is incorporated herein by reference in its entirety.

In some embodiments, during the detection training phase 600, the patch sampler 610 can create randomly selected mini-batches of patches, for instance batches of eight patches, which can be sent forward through the detection module 310. For each patch within the mini-batch, the detection module 310 can compute a single-channel probability map 660 of the same geometry, where each voxel is assigned a probability of belonging to a structure.

In some embodiments, the neural network model implemented in the detection module 310 can be a CNN based on the U-Net architecture, for example as described for instance in Kerfoot, E., Clough, J., Oksuz, I., Lee, J., King, A. P., & Schnabel, J. A. (2018); Left-ventricle quantification using residual U-Net; International Workshop on Statistical Atlases and Computational Models of the Heart, 371-380, the disclosure of which is incorporated herein by reference in its entirety. More specifically, the network architecture can comprise a contraction and expansion path. The contraction path can comprise a plurality of initial convolution operations followed by a plurality of successive pooling blocks, convolutional blocks and dropout blocks. The contraction path can comprise a plurality of upsampling blocks, matching the same number of downsampling blocks in the expansion path. Each downsampling block can contain two residual units, each composed of a convolution, an instance normalization layer and a parametric rectified linear unit activation. For each downsampling block, the convolution operation of the first residual unit can be defined with a stride of 2 voxels, effectively downscaling the input tensor by a factor of 2. On the upsampling path of the network, each block can be defined a transpose convolution of stride 2, upscaling input tensors by a factor of 2, followed by a residual unit. A skip connection can be established between corresponding downsampling block and upsampling block of the same resolution, concatenating channels of tensors from the downsampling path with the upsampling path. It is appreciated that other CNN architectures are also possible. Such architectures can be fully convolutional and comprise a contracting and expanding path connected by multiple skip connections.

In some embodiments, the neural network model implemented in the detection module 310 can be configured with dropout, such that during detection training 600, some connections can be randomly dropped, forcing the model to adopt a different configuration for each training batch. This can have a regularizing effect on the optimization and allows to estimate the model uncertainty at test time.

Neural network architectures such as the U-Net are normally defined by a number of hyperparameters, such as number of layers, size of convolution and dropout rate. In some embodiments, the model hyperparameters can be adjusted through an ablation study to maximize the detection sensitivity.

Loss detection 670 can implement a loss function to compare the probability map 660 generated by the detection module 310 with the ground truth 650. As can be appreciated, any suitable loss function can be used for this purpose. As an example, the Dice loss function can be used, which can be expressed as

$${L}_{D i c e} ( g , p ) = \frac{2 {g}^{T} p + ϵ}{{p}^{T} p + {g}^{T} g + ϵ}$$

where g and p are respectively the ground truth and predicted foreground voxels, and ϵ is any small scalar value suitable to avoid a division by zero. As can be appreciated, while the Dice loss function attributes an equal weight to each voxel forming structures within a batch, it can ignore tiny structures in favour of larger or bigger ones.

Accordingly, in some embodiments, the detection loss 670 can implement a modified Dice loss function, such as a structure-wise Dice loss function, designed for heterogeneous and small structure segmentation, where a Dice score value DSCₛ is computed independently for each separate structure s present in the ground truth. The final loss value is computed as the weighted sum of individual per structure Dice loss components, which can be expressed as:

$${L}_{Struct - D i c e} = {\sum }_{s} {w}_{s} ( 1 - D S {C}_{s}^{1 / β} )$$

where wₛ is a weighting factor capturing prior knowledge about structures, 1/β is a focal factor reducing the contribution of well segmented structures and DSCₛ is the per-structure Dice score which can be expressed as:

$$D S {C}_{s} = \frac{2 {g}_{s}^{T} {p}_{s} + ϵ}{{p}_{s}^{T} {p}_{s} + {g}_{s}^{T} {g}_{s} + ϵ}$$

where gₛ and pₛ are respectively the ground truth and predicted foreground voxels in the vicinity of structure s. In some embodiments, the vicinity of structure s comprises the set of voxels for which the closest structure is structure s. In some embodiments, the weighting factors wₛ are inversely proportional to the volume of each structure s and sums to 1.

As an example, to mitigate the impact of structure size imbalance between small and large structures within the same batch, a modified Dice loss function, such as a volume aware (VA) Dice loss function can be implemented by detection loss 670. In such embodiments, the contribution of individual structures to the Dice loss can be reweighted by the inverse of their volume, emphasizing the loss incurred by missing smaller structures. The VA Dice loss function can be expressed as:

$${L}_{VA} ( g , p ) = \frac{C {g}^{T} Wp + ϵ}{{p}^{T} p + {g}^{T} Wg + ϵ}$$

where g and p are respectively the ground truth and predicted foreground voxels, C is a normalization constant ensuring the loss is bounded between 0 and 1, such as:

$$C = 1 + \frac{{g}^{T} g}{{g}^{T} W g}$$

And where W is a weighting matrix, such as:

$${W}_{i i} = \{ \begin{matrix}\sqrt{\frac{λ}{vol ( i )}} , & vol ( i ) \neq 0 \\ 0 , & otherwise\end{matrix}$$

Where vol(i) is the volume of the structure to which the voxel i belongs, and where parameter λ is set to be the number of voxels of the largest structure in the current batch. In some embodiments, lesions can be reweighted by how well the structure was detected overall. Such a reweighting can take into account not the volume, but the overall classification accuracy of the voxels forming a structure. As an example, the weight matrix can be defined as:

$${W}_{i i} = \{ \begin{matrix}\frac{{\sum }_{i} {( 1 - {p}_{i} )}^{γ}}{vol ( i )} , & vol ( i ) \neq 0 \\ 0 , & otherwise\end{matrix}$$

Where pᵢ is the predicted structure probability for a voxel belonging to structure i. The higher those probabilities are for all voxels of structure i, the lower those voxel will weight in the Dice loss computation.

After each mini-batch has been provided as input to the detection module 310, an optimization step 680 can be performed iteratively to adjust the parameters in order to minimize the loss. Optimization 680 can be performed by applying a stochastic gradient descent algorithm. In some embodiments, modified algorithms such as the adaptive gradient algorithm or the root mean square propagation algorithm can be used for optimization 680. In some embodiments, an adaptive moment estimation (Adam) algorithm can be used, as described in Kingma, D. P., & Ba, J. (2015); Adam: A method for stochastic optimization; 3rd International Conference for Learning Representations, the disclosure of which is incorporated herein by reference in its entirety. An initial learning rate, or α, can be defined and can be configured to be multiplied by a predetermined factor each time the loss has been stable for a predetermined number of epochs, with each epoch corresponding to a predetermined number of patches sampled from each image. As an example, the initial learning rate, can be 10⁻³, and can be configured to be multiplied by a factor of 0.5 each time the loss has been stable for more than 50 epochs, each epoch corresponding to 5 patches being sampled from each image 510, randomly shuffled and queued to train the detection module 310.

After a configurable amount of time has elapsed or a configurable number of epochs have been run, the detection training phase 600 can be halted and a trained model, for instance the model with the lowest loss, can be retained for use in the detection module 310. In embodiments using random dropout, the model can produce a configurable plurality of output probability maps, such that any tensor fed as input to the detection module 310 in production can be passed through the same model several times, generating a plurality of probability maps, for example as shown in the system 103 in FIG. 4.

The contouring training phase 700 can be independent from detection training phase 600 and can therefore be performed before, after or in parallel. At training time, the box sampler 330 can be provided each rasterized segmentation map 570 and brain extracted images 550 as input and generate samples comprising cropped bounding boxes of each initial image 510, where each sample contains a centred and zoomed-in structure, such that each lesion structure is surrounded by a configurable margin, for instance 20 voxels. The samples can then be assembled in mini-batches and provided to the contouring module 340 for training, with the corresponding regions of the signed distance map 580 used as ground truth.

In some embodiments, the input of the contouring module 340 at training time can comprise a two-channel tensor, wherein the first channel includes the image, and the second channel includes the ground truth segmentation. In some embodiments, in order for the model not to rely on the produced segmentation exclusively, the ground truth channel can be randomly dropped i.e., left blank, during training, forcing the model to use it as an unreliable hint only.

The output of the contouring module 340 can be a single-channel tensor representing a distance map 720, where each voxel is assigned a value that represents the distance from the ground truth boundary.

During loss detection 730, a loss function can compare the produced signed distance map 720 and the ground truth distance map 580. In some embodiments, the loss function is an L₂ loss function. In some embodiments, the L₂ loss function can be clamped such that, above and below a configurable distance δ, errors are ignored. This can force the model to focus on optimizing the part of the distance map closest to the target boundary. This can for instance be expressed as

$${L}_{2} ( \hat{s} , s ) = {‖ clamp ( \hat{s} , δ ) - clamp ( s , δ ) ‖}^{2} + λ {‖ w ‖}^{2}$$

where the predicted distance ŝ is compared to the distance map value of the ground truth s, with

$$clamp ( x , δ ) := min ( δ , max ( - δ , x ) )$$

and w is a regularizing term based on the intensity gradients

$$w = ∇ \hat{s} - ∇ s$$

The optimization 740 of the contouring module 340 can be performed in any of the ways that the optimization 680 of the detection module 310 can, as described above.

It will be appreciated that the present disclosure allows implementing a detection and contouring system that can be sensitive to small structures while producing a reasonable number of false positives. The system can be calibrated such that confidence scores match the observed error rate and is able to produce precise lesion contours independent of the underlying image resolution.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A method for detecting and contouring structures of interest in a 3D image, the method comprising:
detecting structures of interest from a multi-channel input comprising at least one 3D image and generating a corresponding segmentation map of the detected structures using a first neural network;
extracting a plurality of cropped images from the at least one 3D image, each cropped image corresponding to a subregion of the at least one 3D image containing at least one of the detected structures contained in the segmentation map; and
estimating contours of the detected structures in the plurality of cropped images and generating corresponding shape representations of the estimated contours using a second neural network.

2. The method according to claim 1, wherein the multi-channel input comprises feature descriptors inferred from the at least one 3D image using a third neural network.

3. The method according to claim 2, wherein the at least one 3D image is a radiographic image of an organ and the feature descriptors comprise anatomical priors.

4. The method according to claim 1, wherein the at least one 3D image is a radiographic image of a brain, and the multi-channel input comprises a brain parenchyma boundary inferred from the at least one 3D image.

5. The method according to claim 1, wherein detecting structures of interest comprises generating the segmentation map by thresholding a probability map corresponding to an inferred probability of each voxel of the at least one 3D image being part of one of the structures of interest.

6. The method according to claim 5, wherein detecting structures of interest comprises at least one of:
performing the steps of applying the first neural network N times using the same multi-channel input with dropout and data augmentation to generate N intermediate probability maps; and aggregating the N intermediate probability maps to generate the probability map; and
performing the steps of sampling the at least one 3D image into patches; applying the first neural network to each of the patches to generate a plurality of corresponding intermediate patch probability maps; and aggregating the intermediate patch probability maps to generate the probability map.

7. (canceled)

8. The method according to claim 6, wherein detecting structures of interest comprises sampling the at least one 3D image, and wherein sampling the at least one 3D image comprises at least one of: sampling the at least one 3D image in a grid-like fashion; and applying a sliding window algorithm, such that regions of the at least one 3D image represented in adjacent patches at least partially overlap.

9. (canceled)

10. The method according to claim 1, further comprising displaying the detected structures and providing controls allowing a user to: approve or reject the detected structures and/or modify contours of the detected structures to generate a new shape representation.

11. The method according to claim 1, wherein the detecting, extracting and estimating are carried out to automatically detect and contour a plurality of structures of interest, further wherein the method comprises receiving a user input corresponding to a manually defined bounding box, extracting an additional cropped image from the at least one 3D image corresponding to a subregion of the at least one 3D image within the bounding box, and estimating contours of an additional structure within the additional cropped image using the second neural network.

12. The method according to claim 1, wherein the first neural network is trained using a modified Dice loss function expressed as:
$$L ( g , p ) = \frac{C {g}^{T} Wp + ϵ}{{p}^{T} p + {g}^{T} W g + ϵ}$$
where g and p are respectively ground truth and predicted foreground voxels, C is a normalization constant ensuring the loss is bounded between 0 and 1, ϵ is a scalar value suitable to avoid a division by zero, and W is a weighting matrix.

13-19. (canceled)

20. A non-transitory computer-readable medium having instructions stored thereon which, when executed by one or more processors of a computing system, cause the computing system to:
detect structures of interest from a multi-channel input comprising at least one 3D image and generate a corresponding segmentation map of the detected structures using a first neural network;
extract a plurality of cropped images from the at least one 3D image, each cropped image corresponding to a subregion of the at least one 3D image containing at least one of the detected structures contained in the segmentation map; and
estimate contours of the detected structures in the plurality of cropped images and generate corresponding shape representations of the estimated contours using a second neural network.

21. A computing system for detecting and contouring structures of interest in a 3D image, the computing system comprising one or more processors and memory, the memory having instructions stored thereon which implement:
a detection module configured to detect structures of interest from a multi-channel input comprising at least one 3D image and generate a corresponding segmentation map of the detected structures using a first neural network;
a box sampler configured to extract a plurality of cropped images from the at least one 3D image, each cropped image corresponding to a subregion of the at least one 3D image containing at least one of the detected structures contained in the segmentation map; and
a contouring module configured to estimate contours of the detected structures in the plurality of cropped images and generate corresponding shape representations of the estimated contours using a second neural network.

22. The system according to claim 21, further comprising a preprocessing module configured to infer feature descriptors from the at least one 3D image using a third neural network, wherein the multi-channel input comprises the feature descriptors.

23. The system according to claim 22, wherein the at least one 3D image is a radiographic image of an organ and the feature descriptors comprise anatomical priors.

24. The system according to claim 21, wherein the at least one 3D image is a radiographic image of a brain, and the multi-channel input comprises a brain parenchyma boundary inferred from the at least one 3D image.

25. The system according to claim 21, wherein the box sampler is configured to generate the segmentation map by thresholding a probability map corresponding to an inferred probability of each voxel of the at least one 3D image being part of one of the structures of interest.

26. The system according to claim 25, wherein the detection module is configured to perform at least one of:
performing the steps of applying the first neural network N times using the same multi-channel input with dropout and data augmentation to generate N intermediate probability maps; and aggregating the N intermediate probability maps to generate the probability map; and
performing the steps of sampling the at least one 3D image into patches; applying the first neural network to each of the patches to generate a plurality of corresponding intermediate patch probability maps; and aggregating the intermediate patch probability maps to generate the probability map.

27. The system according to claim 26, wherein the detection module is configured to perform sampling of the at least one 3D image, and wherein sampling the at least one 3D image comprises at least one of: sampling the at least one 3D image in a grid-like fashion; and applying a sliding window algorithm, such that regions of the at least one 3D image represented in adjacent patches at least partially overlap.

28. The system according to claim 21, further comprising an interactive validation module configured to display the detected structures and provide controls allowing a user to: approve or reject the detected structures and/or modify contours of the detected structures to generate a new shape representation.

29. The system according to claim 21, further comprising a graphical user interface configured to receive a user input corresponding to a manually defined bounding box, wherein the box sampler is configured to extract an additional cropped image from the at least one 3D image corresponding to a subregion of the at least one 3D image within the bounding box, and wherein the contouring module is configured to estimate contours of an additional structure within the additional cropped image using the second neural network.
